(19) United States
(12) Patent Application Publication
(10) Pub. No.: US 2019/0079280 A1
(43) Pub. Date: Mar. 14, 2019

(54) MANUFACTURING METHOD OF OPTICAL UNIT FOR ENDOSCOPE, OPTICAL UNIT FOR ENDOSCOPE, AND ENDOSCOPE

(71) Applicant: OLYMPUS CORPORATION, Tokyo (JP)

(72) Inventors: Kazuhiro YOSHIDA, Okaya-shi (JP); Takatoshi IGARASHI, Ina-shi (JP); Kazuya MAEDA, Kamiina-gun (JP)

(73) Assignee: OLYMPUS CORPORATION, Tokyo (JP)

(21) Appl. No.: 16/185,087

(22) Filed: Nov. 9, 2018

Related U.S. Application Data
(63) Continuation of application No. PCT/JP2016/065281, filed on May 24, 2016.

(51) Int. Cl. G02B 23/24; H04N 5/225; A61B 1/05; A61B 1/00; H01L 27/146

(52) U.S. Cl. CPC G02B 23/2423; H04N 5/2253; A61B 1/051; A61B 1/0011; A61B 1/00096; H01L 27/14636; H01L 27/14618; H01L 27/14625; H01L 27/14685; H04N 2005/2255; G02B 23/2484

(57) ABSTRACT

A manufacturing method of the optical unit for endoscope includes: a process of fabricating a bonded wafer by laminating a plurality of optical element wafers, each of the plurality of optical element wafers including a plurality of optical elements; a groove forming process of forming a groove on the bonded wafer along a cutting line for segmentation such that the groove has a bottom surface in the optical element wafer laminated at a lowermost part of the bonded wafer; and a cutting process of cutting the bonded wafer along the cutting line with a cutting margin narrower than a width of the groove and segmenting the bonded wafer, and the manufacturing method further includes a process of disposing a reinforcing member in the groove.

### CROSS REFERENCE TO RELATED APPLICATION

This application is a continuation application of PCT/JP2016/065281 filed on May 24, 2016, the entire contents of which are incorporated herein by this reference.

### BACKGROUND OF THE INVENTION

### Field of the Invention

The present invention relates to a manufacturing method of an optical unit for endoscope configured by laminating a plurality of optical elements, the optical unit for endoscope configured by laminating the plurality of optical elements, and an endoscope including, at a distal end portion thereof, the optical unit for endoscope configured by laminating the plurality of optical elements.

### Background Art

In an optical unit for endoscope to be disposed at a distal end portion of an endoscope, size reduction, in particular, reduction in the diameter size is significant for achieving low invasion.

Japanese Patent Application Laid-Open Publication No. 2012-18993 discloses a manufacturing method of an optical unit constituted of a wafer-level laminated body, as a method of efficiently manufacturing an extremely slim optical unit. The optical unit is fabricated by cutting a bonded wafer formed by laminating a plurality of lens wafers each including a plurality of lenses and an image pickup device wafer including a plurality of image pickup devices and by segmenting the bonded wafer into wafer-level laminated bodies.

### SUMMARY OF THE INVENTION

A manufacturing method of an optical unit for endoscope according to an embodiment of the present invention includes: a process of fabricating a bonded wafer by laminating a plurality of optical element wafers, each of the plurality of optical element wafers including a plurality of optical elements; a groove forming process of forming a groove on the bonded wafer along a cutting line for segmentation such that the groove has a bottom surface in the optical element wafer laminated at a lowermost part of the bonded wafer; and a cutting process of cutting the bonded wafer along the cutting line with a cutting margin narrower than a width of the groove and segmenting the bonded wafer, and the manufacturing method further includes a process of disposing a reinforcing member in the groove.

An optical unit for endoscope according to an embodiment of the present invention includes a plurality of optical elements including a first optical element, the first optical element including a first principal surface as a light incident surface and a second principal surface opposed to the first principal surface, the first optical element having a base body made of a parallel flat glass, the plurality of optical elements being laminated and configured to form an image of light entered from the light incident surface, wherein a recess is formed on a side surface of the optical unit, the recess allowing the first principal surface to be larger than any of principal surfaces of the plurality of optical elements other than the first optical element, the recess being formed to a halfway position of a side surface of the first optical element, and a reinforcing member is housed inside the recess.

An endoscope according to an embodiment of the present invention includes an optical unit for endoscope at a distal end portion of an insertion portion, the optical unit for endoscope including: a plurality of optical elements including a first optical element, the first optical element including a first principal surface as a light incident surface and a second principal surface opposed to the first principal surface, the first optical element having a base body made of a parallel flat glass, the plurality of optical elements being laminated and configured to form an image of light entered from the light incident surface, wherein a recess is formed on a side surface of the optical unit, the recess allowing the first principal surface to be larger than any of principal surfaces of the plurality of optical elements other than the first optical element, the recess being formed to a halfway position of a side surface of the first optical element, and a reinforcing member is housed inside the recess.

### BRIEF DESCRIPTION OF THE DRAWINGS

FIG. 1 is a perspective view of an endoscope according to an embodiment.

FIG. 2 is a perspective view of an optical unit according to a first embodiment.

FIG. 3 is a cross-sectional view taken along the line III-III in FIG. 2 of the optical unit according to the first embodiment.

FIG. 4 is an exploded view of the optical unit according to the first embodiment.

FIG. 5 is a flowchart for describing a manufacturing method of the optical unit according to the first embodiment.

FIG. 6 is an exploded view for describing the manufacturing method of the optical unit according to the first embodiment.

FIG. 7 is a perspective view of a bonded wafer, for describing the manufacturing method of the optical unit according to the first embodiment.

FIG. 8 is a perspective view of a groove forming process, for describing the manufacturing method of the optical unit according to the first embodiment.

FIG. 9 is a cross-sectional view of the groove forming process, for describing the manufacturing method of the optical unit according to the first embodiment.

FIG. 10 is a cross-sectional view of a reinforcing member disposing process, for describing the manufacturing method of the optical unit according to the first embodiment.

FIG. 11 is a cross-sectional view of a cutting process, for describing the manufacturing method of the optical unit according to the first embodiment.

FIG. 12 is a cross-sectional view of a cutting process, for describing a manufacturing method of an optical unit according to a modified example 1 of the first embodiment.

FIG. 13 is a cross-sectional view of an optical unit according to a modified example 2 of the first embodiment.

FIG. 14 is a bottom plan view of an optical unit according to a modified example 3 of the first embodiment.

FIG. 15 is a cross-sectional view for describing a manufacturing method of an optical unit according to a second embodiment.

FIG. 16 is a perspective view for describing a manufacturing method of an optical unit according to a third embodiment.

### DETAILED DESCRIPTION OF THE PREFERRED EMBODIMENT(S)

### First Embodiment

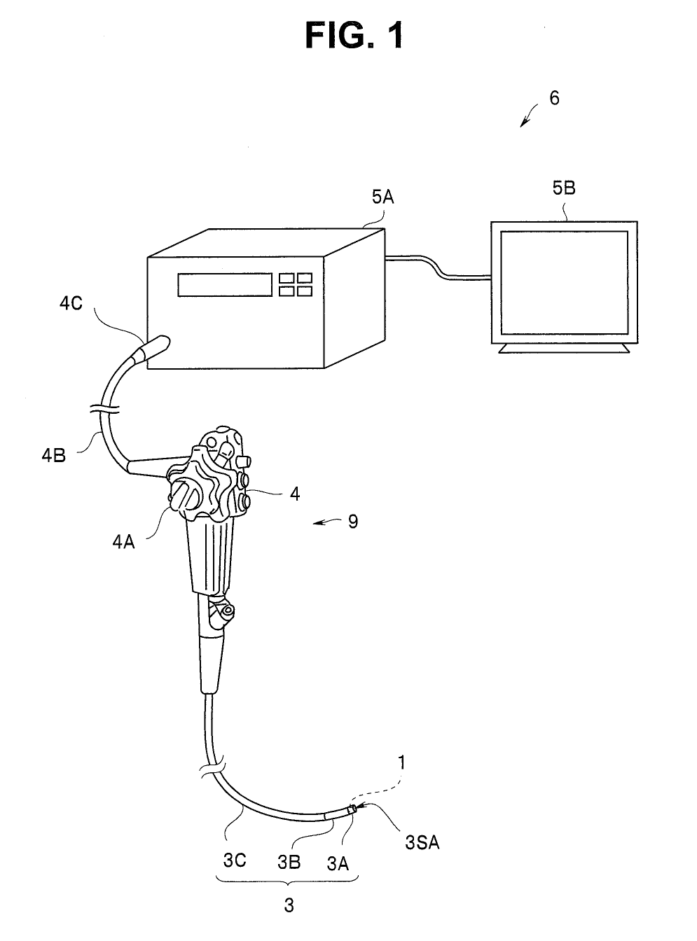

As shown in FIG. 1, an optical unit for endoscope 1 (hereinafter, also referred to as “optical unit 1”) according to the present embodiment is disposed at a distal end portion 3A of an insertion portion 3 of an endoscope 9.

Note that each of the drawings based on each of the embodiments is a pattern diagram in the description below, and care should be taken to the fact that the relationship between the thicknesses and widths of the respective parts, a ratio of the thicknesses and relative angles of the respective parts, and the like are different from the actual ones, and there is a case where the respective drawings include parts in which the relationships and ratios among the dimensions of the respective parts are different from those in other drawings. In addition, there is a case where illustration of some constituent elements is omitted.

An endoscope 9 includes an insertion portion 3, a grasping portion 4 disposed on a proximal end portion side of the insertion portion 3, a universal cord 4B extended from the grasping portion 4, and a connector 4C disposed on a proximal end portion side of the universal cord 4B. The insertion portion 3 includes a distal end portion 3A at which the optical unit 1 is disposed, a bending portion 3B extended from the proximal end side of the distal end portion 3A and configured to be bendable and to change a direction of the distal end portion 3A, and a flexible portion 3C extended from the proximal end side of the bending portion 3B. The optical unit 1 includes a light incident surface 10SA exposed on a distal end surface 3SA of the distal end portion 3A (see FIG. 3). The grasping portion 4 is provided with an angle knob 4A configured to rotate. The angle knob 4A is an operation portion configured to be operated by an operator to operate the bending portion 3B.

The universal cord 4B is connected to the processor 5A through a connector 4C. The processor 5A is configured to control an entire endoscope system 6, perform signal processing on an image pickup signal outputted from the optical unit 1, and output the image pickup signal subjected to the signal processing as an image signal. A monitor 5B displays the image signal outputted from the processor 5A as an endoscopic image. Note that the endoscope 9 is a flexible endoscope. However, the endoscope 9 may be a rigid endoscope as long as the endoscope includes a bending portion. That is, the flexible portion and the like are not essential constituent elements of the endoscope according to the embodiment.

### <Configuration of Optical Unit>

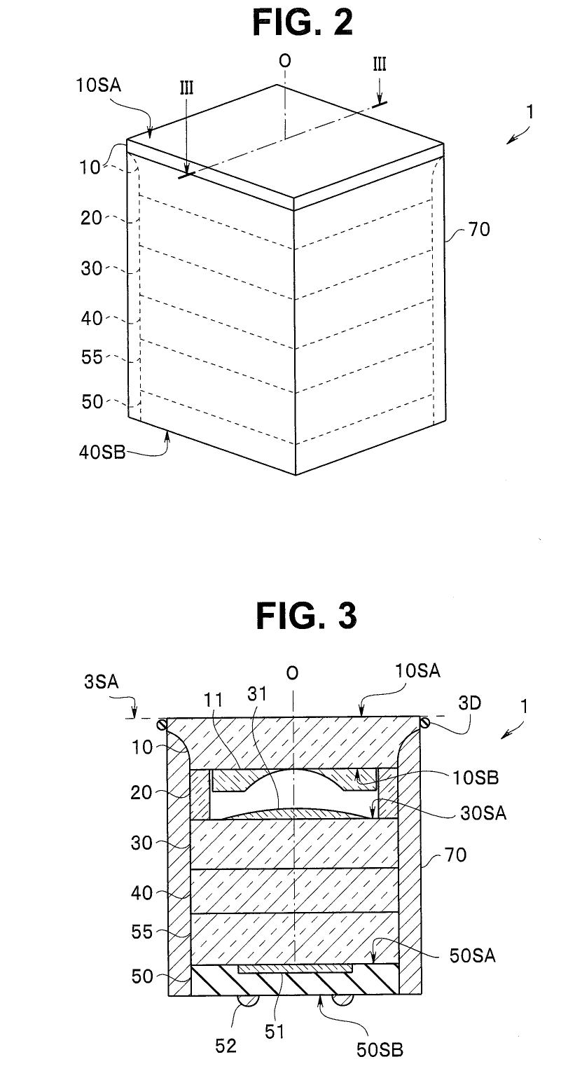

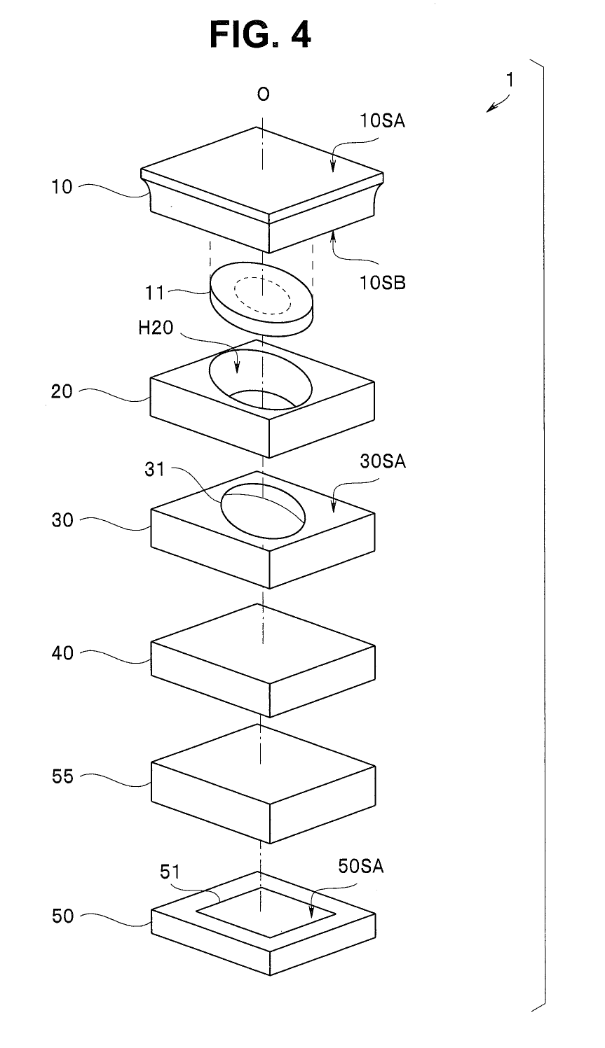

As shown in FIGS. 2 to 4, the optical unit for endoscope 1 is a laminated body formed by laminating a plurality of optical elements 10 to 55 including an image pickup device 50. The plurality of optical elements 10 to 55 are bonded to one another with a resin adhesive, for example, an ultraviolet curable resin disposed between the respective optical elements, though not shown.

The first optical element 10 arranged on the uppermost part of the laminated body includes a first principal surface 10SA as a light incident surface, and a second principal surface 10SB opposed to the first principal surface 10SA. The optical element 10, a base body of which is made of a parallel flat glass, is a hybrid lens element including a resin lens 11 having a negative power which is disposed on the second principal surface 10SB.

A second optical element 20 is a spacer element including, at the center thereof, a through hole which serves as an optical path. The second optical element 20 made of silicon, for example, defines a distance between the first optical element 10 and a third optical element 30.

The third optical element 30, a base body of which is made of a parallel flat glass, is a hybrid lens element including a resin lens 31 having a positive power which is disposed on a third principal surface 30SA. A fourth optical element 40 is an infrared ray cut filter element.

A fifth optical element 55 is a cover glass element 55 configured to protect the image pickup device 50. The image pickup device 50 arranged at the lowermost (rearmost) part of the laminated body includes a light-receiving surface 50SA and a rear surface 50SB opposed to the light-receiving surface 50SA. On the light-receiving surface 50SA, a light-receiving portion 51 such as CMOS light-receiving element is formed. On the rear surface 50SB, a plurality of electrodes 52 connected to the light-receiving portion 51 through through-wiring (not shown) are disposed. The image pickup device 50 receives a driving signal and transmits an image pickup signal through wiring connected to the respective plurality of electrodes 52.

The optical unit 1 is disposed such that the first principal surface 10SA is exposed on the distal end surface 3SA of the distal end portion 3A of the endoscope 9. The optical unit 1 is configured such that the plurality of optical elements 10 to 40 form an image of the light entered from the light incident surface 10SA on the light-receiving portion 51 of the image pickup device 50.

Note that the optical unit 1 includes other optical elements such as a flare diaphragm and a brightness diaphragm, though not shown. In addition, the configuration of the optical unit is not limited to the configuration of the optical unit 1. The configuration such as the numbers of the resin lenses, spacers, and the diaphragms may be appropriately selected according to the specifications of the optical unit.

The optical unit 1 includes recesses (recessed portions) respectively on the four side surfaces. Such recesses allow the first principal surface 10SA to be larger than any of the principal surfaces of other plurality of optical elements 20 to 50, for example, larger than the light-receiving surface 50SA of the image pickup device 50. In other words, each of the recesses is formed from the side surface of the image pickup device 50 serving as a rear end surface of the optical unit 1 to a halfway position of (a part of) the side surface of the first optical element 10.

A reinforcing member 70 is housed inside the recesses on the side surfaces of the optical unit 1. In this configuration, the term “housed” means that the entirety of the reinforcing member 70 is located inside the recesses and the size (external size) of all the cross sections of the reinforcing member 70, which are taken along the direction orthogonal to the optical axis, are equal to or smaller than the size of the first principal surface 10SA.

Note that, in the optical unit 1, since the reinforcing member 70 completely fills the inside of the recesses, the size of all the cross sections of the reinforcing member 70, which are taken along the direction perpendicular to the optical axis, is equal to the size of the first principal surface 10SA. The optical unit 1 configured such that the recesses are filled with the reinforcing member 70 has a rectangular parallelepiped shape same as that of an optical unit which does not include recesses.

In the optical unit 1, the reinforcing member 70 composed of an epoxy resin, for example, is disposed in the recesses on the side surfaces, which improves the mechanical strength of the optical unit. The optical unit 1 is formed to be extremely slim, with the first principal surface 10SA being 1 mm square, for example. Even if a stress is applied to the optical unit which is a segmented piece, the optical unit 1 will not suffer a damage caused by the bonded surface being peeled off or broken, which leads to high productivity. In addition, the reinforcing member 70 is housed in the recesses, which prevents the size increase due to the disposition of the reinforcing member 70 on outer circumference of the optical unit 1. As a result, the extremely slim optical unit 1 can be obtained.

Note that, if the recesses are formed over the entirety of the side surfaces, merely the external size of the optical unit becomes small. In contrast, in the optical unit 1, the recesses are formed so as not to reach the first principal surface of the first optical element 10. In addition, the optical unit 1 is configured such that only the first principal surface 10SA of the first optical element 10 made of glass is exposed outside the distal end surface 3SA of the distal end portion 3A of the endoscope 9. For example, the optical unit 1 is sealed by an O-ring 3D (see FIG. 3) which contacts the side surfaces of the first optical elements 10. Therefore, not only the side surfaces of the resin adhesion layers (not shown) that bond the optical elements 10 to 50 with one another but also the reinforcing member 70 is not exposed outside. Such a configuration prevents water vapor and the like from entering the optical path through the reinforcing member 70 or the interface between the reinforcing member 70 and the optical elements. As a result, the optical unit 1 is excellent in reliability.

Note that it is preferable to use various kinds of hard materials, for example, a material having Vickers hardness of Hv≥5 GPa as a material of the reinforcing member 70, in order to ensure the mechanical strength of the optical unit. Instead of the hard resin, a metal material such as Cu, Ni, or Au, or an inorganic material such as silicon oxide or silicon nitride may be used as the material of the reinforcing member 70.

In addition, in order to prevent external light from entering the optical path, it is particularly preferable to use a resin material including black particles or a metal material as the material of the reinforcing member 70.

It is needless to say that the endoscope 9 including the optical unit 1 at the distal end portion 3A has a small diameter and high productivity.

### <Manufacturing Method of Optical Unit>

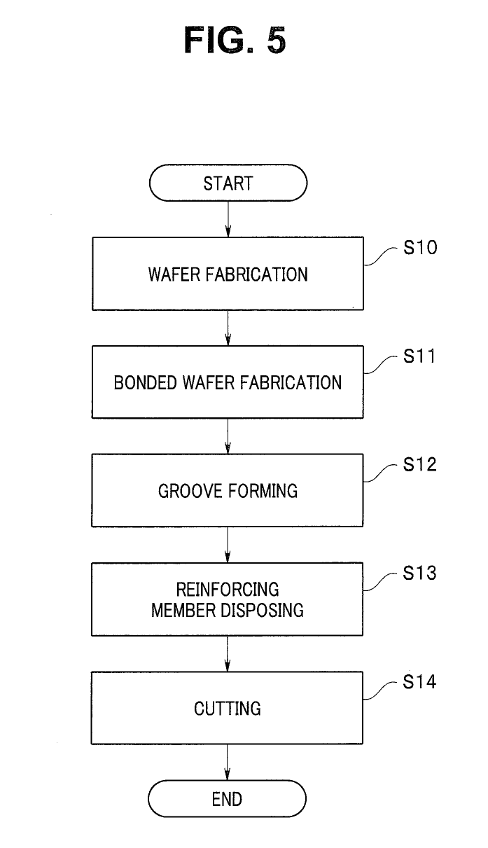

Next, the manufacturing method of the optical unit according to the embodiment will be described referring to the flowchart in FIG. 5. The optical unit 1 is a wafer-level optical unit manufactured by cutting and segmenting a bonded wafer 60W (see FIG. 8) formed by laminating a plurality of element wafers each including a plurality of optical elements arranged in a matrix form and by bonding the plurality of element wafers.

### <Step S10> Wafer Fabrication Process

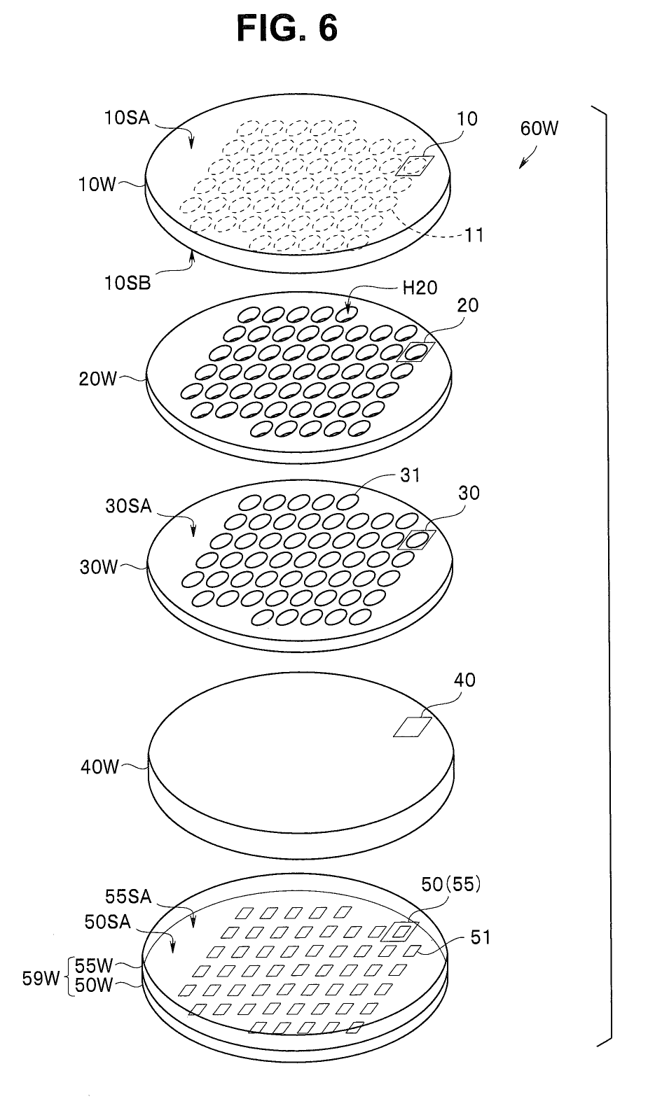

As shown in FIG. 6, a plurality of optical element wafers 10W to 59W respectively including the plurality of optical elements 10 to 51 are fabricated.

The element wafer 10W on which the plurality of first optical elements 10 are arranged is fabricated by disposing the resin lenses 11 each having the negative power on the second principal surface 10SB of the parallel flat glass wafer as the base body of the element wafer 10W. It is preferable to use an energy curable resin as a material of the resin lenses 11. The parallel flat glass wafer is satisfactory if the parallel flat glass wafer is transparent in the wavelength band of the light with which image pickup is performed, and borosilicate glass, quartz glass, or single-crystal sapphire is used, for example.

The energy curable resin receives energy such as heat, ultraviolet ray, or electron ray from outside, and thereby crosslinking reaction or polymerization reaction of the resin progresses. The curable resin is a transparent ultraviolet curable type silicone resin, epoxy resin, or acrylic resin, for example. Note that “transparent” means that the material has such small optical absorption and small light scattering as to allow withstanding for use in the range of the wavelength to be used.

The resin lenses 11 are fabricated by disposing uncured liquid or gel-like ultraviolet curable resin on the glass wafer, and applying ultraviolet ray to cure the resin, with a mold, which has concave portions having a predetermined inner surface shape, being pressed against the resin. Note that, in order to improve the interfacial bond strength of the glass and resin, it is preferable to apply silane coupling treatment and the like to the glass wafer before disposing the resin on the glass wafer. The inner surface shape of the mold is transferred to the outer surface shape of the resin lens, which enables an aspherical lens to be easily fabricated.

The element wafer 20W on which a plurality of second optical elements 20 are arranged is fabricated by forming a plurality of through holes H20 on a silicon wafer by an etching method, for example. Note that, instead of the element wafer 20W, when disposing the resin lenses 11 on the element wafer 10W, for example, a spacer may be simultaneously disposed around the resin lenses 11 using an energy curable resin.

The element wafer 30W on which the third optical elements 30 are arranged is fabricated by disposing the resin lenses 31 each having the positive power on one surface (the third principal surface 30SA) of the parallel flat glass wafer by a method same as the method applied to the element wafer 10W.

The element wafer 40W including a filter as a fourth optical element 40 is a parallel plate wafer made of a filter material. The element wafer 40W is regarded as including a plurality of filter elements 40. The element wafer 40W is a filter wafer made of an infrared ray cut material that removes unnecessary infrared ray (light of wavelength of 770 nm or over, for example). Note that a plate glass wafer including, on a surface thereof, a band-pass filter that transmits only the light of a predetermined wavelength and cut the light of unnecessary wavelength may be used as the filter wafer.

The image pickup device wafer 50W made of a silicon wafer includes a plurality of image pickup devices 50 each including, on a light-receiving surface 50SA, a light-receiving portion 51 formed by a known semiconductor manufacturing technique. The electrodes 52 connected to the light-receiving portion 51 through the through-wiring (not shown) are disposed on the rear surface 50SB of each of the image pickup devices 50. The image pickup device wafer 50W may include a reading circuit.

The element wafer 55W including a cover glass element as a fifth optical elements 55 is a parallel flat glass wafer. The element wafer 55W is regarded as including a plurality of cover glass elements.

Note that, after the element wafer 55W that protects the light-receiving surface 50SA is bonded on the image pickup device wafer 50W through a transparent adhesive resin, the electrodes 52 may be disposed on the rear surface 50SB. In this case, an element wafer 59W is fabricated, in which the element wafer 55W is disposed on the light-receiving surface 50SA of the image pickup device wafer 50W.

### <Step S11> Bonded Wafer Fabrication Process

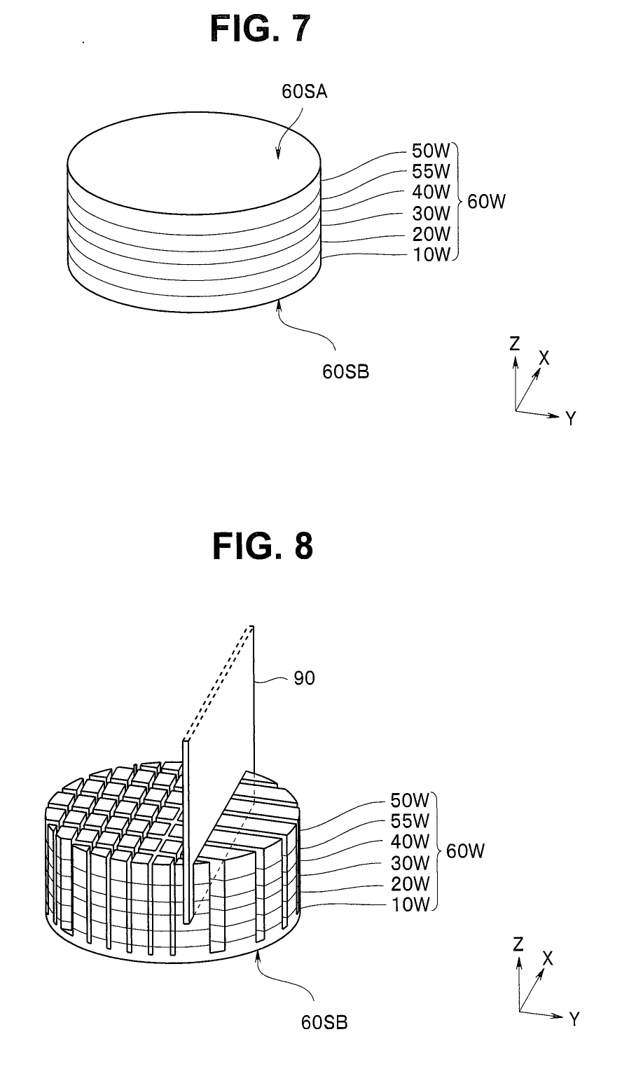

As shown in FIG. 7, the bonded wafer 60W is fabricated by laminating and bonding the plurality of element wafers 10W to 59W. The plurality of element wafers 10W to 59W are bonded to one another with a resin adhesive, for example, an ultraviolet curable resin, not shown. At the uppermost part of the bonded wafer 60W, the image pickup device wafer 50W including the plurality of image pickup devices 50 is laminated.

### <Step S12> Groove Forming Process

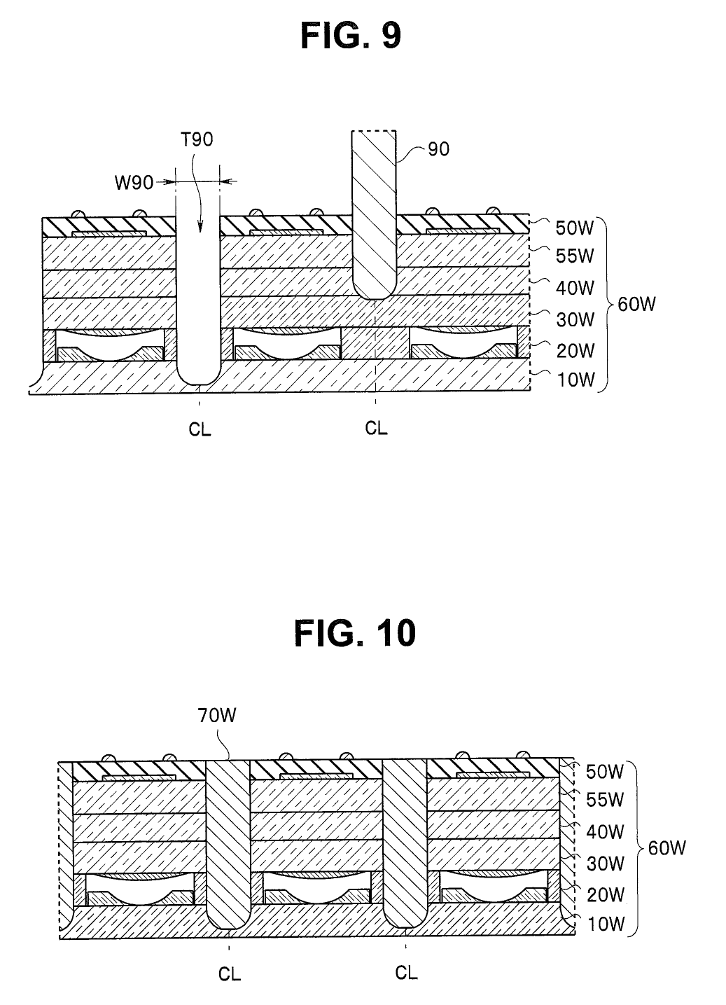

As shown in FIGS. 8 and 9, the first principal surface 10SA of the first element wafer 10W of the bonded wafer 60W is fixed to a dicing tape, for example. Then, grooves T90 are formed on the bonded wafer 60W along the cutting lines CL for segmentation. That is, the grooves T90 each having an opening on the rear surface 50SB of the image pickup device wafer 50W of the bonded wafer 60W are formed.

Note that the cutting lines CL are used for segmenting the bonded wafer 60W into the optical units 1, and include a plurality of lines orthogonal to one another. The respective optical elements 10 to 50 are located in a region surrounded by the four cutting lines.

Each of the grooves T90 is formed along the cutting lines for segmentation by using a first dicing blade 90 having a width of W90, for example, such that each of the grooves has an opening width W90 and has a bottom surface in the first element wafer 10W which is the optical element wafer laminated at the lowermost part of the bonded wafer 60W. When the thickness of the first element wafer 10W is 200 μm, for example, the grooves T90 are formed up to the position of half of the thickness of the first element wafer 10W. Note that the grooves may be formed not by the machining method but by the etching method or the like.

### <Step S13> Reinforcing Member Disposing Process

As shown in FIG. 10, a reinforcing member 70W is filled in the grooves T90 of the bonded wafer 60W. For example, the epoxy resin is filled in the grooves T90 by an ink jet method, for example. Alternatively, an epoxy resin which is a light-shielding material in which carbon particles are dispersed may be filled in the grooves T90. When a plated film is used as the reinforcing member 70W, an insulation layer made of SiO₂ and a base conductive film are disposed in each of the grooves T90, and thereafter copper or the like is disposed using via-fill plating method. In this case, the rear surface 50SB of the image pickup device wafer 50W is covered with a protective resist or the like, in advance. When the reinforcing member 70W is made of a copper plated film, the disposition of the electrodes 52 on the rear surface 50SB of the image pickup device wafer 50W and the disposition of the reinforcing member 70W may be performed at the same time.

### <Step S14> Cutting Process

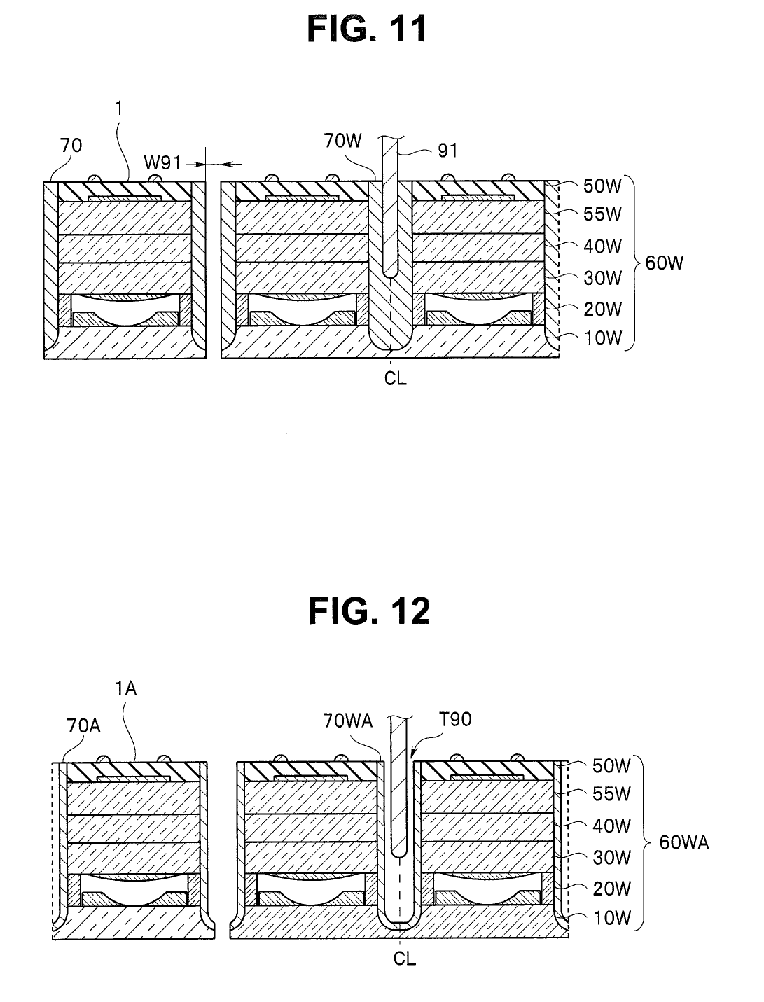

As shown in FIG. 11, the bonded wafer 60W is cut along the cutting lines CL and segmented into a plurality of optical units 1. The width of a cutting margin (the part to be lost by cutting) is W91. That is, the bonded wafer 60W is cut by a second dicing blade 91 having a width 91W. The width W91 of the cutting margin is smaller than the width W90 of each of the grooves. Therefore, the cut surface of the bonded wafer 60W, that is, the side surface of the optical unit 1 is constituted of the cut surface of a part of the first element wafer 10W and the cut surface of the reinforcing member 70. The cutting may be performed with laser dicing or plasma dicing.

With the manufacturing method according to the present embodiment, it is possible to efficiently manufacture the optical unit for endoscope, in which a plurality of optical elements 10 to 55 configured to form an image of the light entered from the light incident surface 10SA are laminated, the light incident surface 10SA is formed to be larger than any of the principal surfaces of the plurality of other optical elements due to the recesses formed on the side surfaces of the optical unit, and the reinforcing member 70 is housed (filled) inside the recesses.

That is, with the manufacturing method of the present embodiment, it is possible to efficiently manufacture the extremely slim optical unit 1 whose mechanical strength is improved by the reinforcing member 70 and which has high productivity.

In addition, the manufacturing method of the present embodiment enables the extremely slim optical unit 1 including on the side surfaces thereof the light-shielding material to be efficiently manufactured.

### Modified Examples of First Embodiment

Next, optical units 1A to 1C according to the modified examples of the first embodiment will be described. The optical units 1A to 1C are similar to the optical unit 1 and have the same effect as that of the optical unit 1. The same constituent elements are attached with the same reference numerals and descriptions thereof will be omitted.

### Modified Example 1 of First Embodiment

As shown in FIG. 12, in the optical unit 1A according to the present modified example, a reinforcing member 70WA is coated on the wall surface (inner surface) of each of the grooves T90 and the inside of each of the grooves T90 is not fully filled with the reinforcing member 70WA in a bonded wafer 60WA.

For example, an inorganic material made of silicon oxide or silicon nitride, or a metal material, which is coated by the sputtering method, the CVD method, or the plating method, is more excellent in mechanical strength than the resin material. Therefore, although the reinforcing member 70A does not fill the inside of the recesses on the side surfaces of the optical unit, the mechanical strength of the optical unit is ensured. Note that the reinforcing member 70A preferably has a light-shielding property.

The film thickness of the reinforcing member 70A is preferably 1 μm or more, and more preferably 5 μm or more. If the film thickness is within the above-described range, the mechanical strength is ensured. Furthermore, if the film thickness of the reinforcing member 70A is 10 μm or more, the light shielding property is also ensured. The upper limit of the film thickness of the reinforcing member 70A is less than half of the width W90 of the grooves T90.

The bonded wafer 60WA can be cut more easily than the bonded wafer 60W. In addition, the optical unit 1A is allowed to include another member in the space which is created in each of the recesses on the side surfaces and in which the reinforcing member 70A is not filled.

### Modified Example 2 of First Embodiment

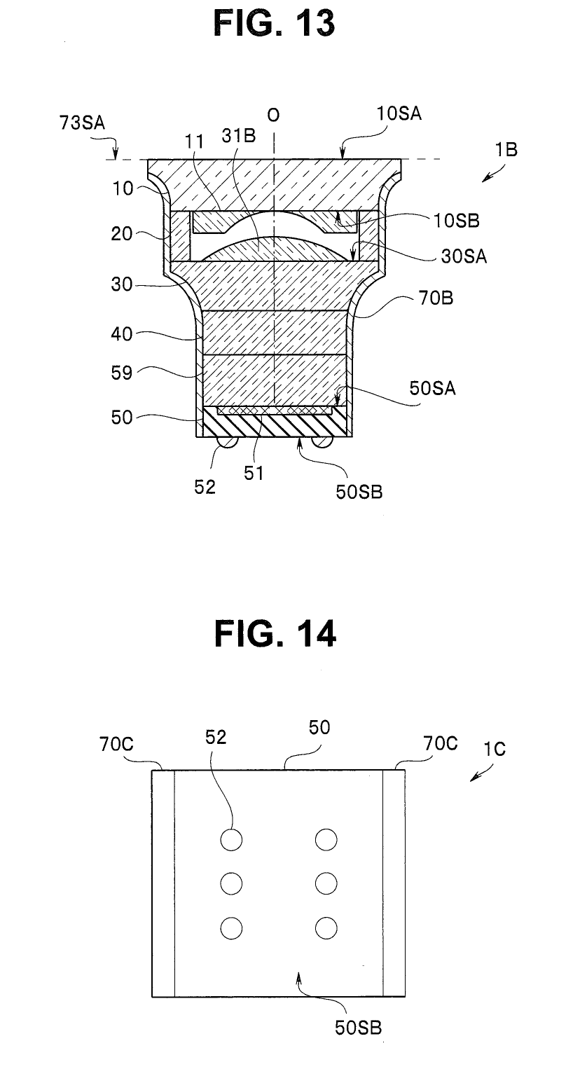

As shown in FIG. 13, the optical unit 1B is configured such that the recesses formed on the side surfaces of the optical unit have two stages of depth. The optical unit 1B is manufactured as follows. A first groove is formed on the bonded wafer, and then a second groove having a width narrower than that of the first groove and a depth deeper than that of the first groove is formed on the bonded wafer, and thereafter the bonded wafer is segmented into optical units 1B. It is needless to say that, after the second groove is formed on the bonded wafer, the first groove having the width wider than that of the second groove and the depth shallower than that of the second groove may be formed.

In the optical unit 1B, the size of the light-receiving surface 50SA of the image pickup device 50 is smaller than the size of the second principal surface 10SB of the first optical element 10.

In a wide angle optical unit, there is a case where the area of the optical path on the image pickup device 50 side becomes smaller than the area of the first principal surface 10SA. Therefore, the recesses having two stages of depth can be formed. The optical unit 1B is allowed to include more other members in the space which is created in each of the recesses and in which the reinforcing member 70B is not filled.

### Modified Example 3 of First Embodiment

As shown in the bottom plan view (the rear surface 50SB of the image pickup device 50) in FIG. 14, the optical unit 1C includes a reinforcing member 70C only on two side surfaces opposed to each other.

That is, as long as the mechanical strength of the optical unit is ensured, the reinforcing member does not have to be disposed on all of the four side surfaces as in the optical unit 1. In addition, the optical unit may be configured such that the reinforcing member may be disposed on one side surface or on the three side surfaces of the optical unit.

Note that the optical unit may be formed in a polygonal column shape, the side surfaces of which are chamfered and the cross section of which has a hexagonal shape, or in a columnar shape in the cutting process, or by the processing after the cutting process. In addition, also in the optical unit formed in the above-described shape, the reinforcing member does not have to be disposed on all of the side surfaces.

### Second Embodiment

An optical unit 1D for endoscope according to the second embodiment is similar to the optical units 1 to 1C and has the same effects as those of the optical units 1 to 1C. The same constituent elements are attached with the same reference numerals and descriptions thereof will be omitted.

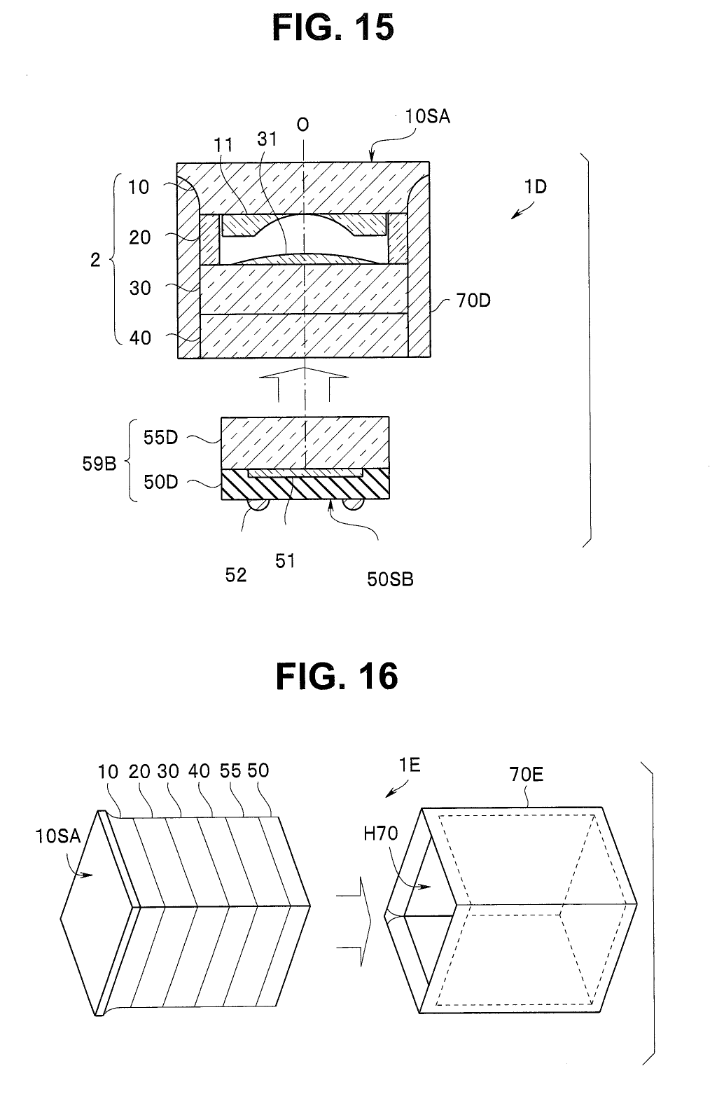

As shown in FIG. 15, the optical unit 1D for endoscope according to the second embodiment is manufactured by bonding a wafer-level image pickup optical system 2 and an image pickup device unit 59B.

That is, in the manufacturing method of the optical unit 1D, the bonded wafer does not include the element wafer 55W as the element wafer and the image pickup device wafer 50W. In addition, the image pickup device unit 59B is manufactured by cutting the image pickup device wafer 50W to which the element wafer 55W as the element wafer is bonded.

For example, the optical unit 1D is manufactured by bonding the wafer-level image pickup optical system 2 and the image pickup device unit 59B that are both determined as good products in the inspection. Therefore, such a manufacturing method eliminates the possibility that one of the wafer-level image pickup optical system or the image pickup device unit is a defect product, and thereby prevents the wafer-level image pickup optical system 2 and the image pickup device unit 59B bonded to each other from being wasted. As a result, the optical unit 1D can be effectively produced at a lower cost than the cost of the optical unit 1 and the like.

### Third Embodiment

An optical unit for endoscope 1E according to the third embodiment is similar to the optical units 1 to 1D and has the same effects as those of the optical units 1 to 1D. The same constituent elements are attached with the same reference numerals and descriptions thereof will be omitted.

As shown in FIG. 16, in the optical unit 1E, a reinforcing member 70E is a lens frame made of a metal frame, for example. That is, the reinforcing member 70E is a hollow rectangular column, and a wafer-level optical unit including recesses on the side surfaces is inserted in the hollow portion. The reinforcing member 70E may be fixed to the wafer-level optical unit by an elastic force or through a resin adhesion layer.

The optical unit 1E is provided with the reinforcing member 70E whose outer periphery of the side surfaces serves as a lens frame for reinforcement and light shielding. However, the thickness of the reinforcing member 70E is set to be equal to or less than the depth of the recesses on the side surfaces. That is, since the reinforcing member 70E disposed on the side surfaces is housed in the recesses, optical unit 1E is excellent in the mechanical strength, even though the optical unit is extremely slim.

In addition the distal end surfaces of the recesses (wall surfaces of the grooves of the first optical element 10) are brought into contact with the distal end of the reinforcing member 70E, which enables the reinforcing member 70E to be arranged precisely at a predetermined position. That is, the recesses are used also as erection of reference points of the reinforcing member 70E.

Note that it is needless to say that the endoscopes including at the distal end portion thereof the optical units 1A to 1E have the same effect as that of the endoscope 9 including the optical unit 1, and further include the effects of the respective optical units 1A to 1E.

The present invention is not limited to the above-described embodiments, but various changes, modifications, and the like are possible without changing the gist of the present invention.

What is claimed is:

1. A manufacturing method of an optical unit for endoscope comprising:
a process of fabricating a bonded wafer by laminating a plurality of optical element wafers, each of the plurality of optical element wafers including a plurality of optical elements;
a groove forming process of forming a groove on the bonded wafer along a cutting line for segmentation such that the groove has a bottom surface in the optical element wafer laminated at a lowermost part of the bonded wafer; and
a cutting process of cutting the bonded wafer along the cutting line with a cutting margin narrower than a width of the groove and segmenting the bonded wafer,
the manufacturing method further comprising a process of disposing a reinforcing member in the groove.

2. The manufacturing method of the optical unit for endoscope according to claim 1, wherein the optical element wafer laminated at the lowermost part of the bonded wafer is a first optical element wafer having a base body made of a parallel flat glass wafer.

3. The manufacturing method of the optical unit for endoscope according to claim 2, wherein the process of disposing the reinforcing member is a process of disposing the reinforcing member in the groove of the bonded wafer, the process of disposing the reinforcing member being performed before the cutting process.

4. The manufacturing method of the optical unit for endoscope according to claim 3, wherein the reinforcing member is made of a light-shielding material.

5. The manufacturing method of the optical unit for endoscope according to claim 4, wherein the reinforcing member is made of a resin material filled in the groove.

6. The manufacturing method of the optical unit for endoscope according to claim 3, wherein the reinforcing member is an inorganic material film or a plated film coated on an inner surface of the groove.

7. The manufacturing method of the optical unit for endoscope according to claim 1, wherein an image pickup device wafer including a plurality of image pickup devices is laminated at an uppermost part of the bonded wafer.

8. An optical unit for endoscope comprising:
a plurality of optical elements including a first optical element, the first optical element including a first principal surface as a light incident surface and a second principal surface opposed to the first principal surface, the first optical element having a base body made of a parallel flat glass, the plurality of optical elements being laminated and configured to form an image of light entered from the light incident surface,
wherein a recess is formed on a side surface of the optical unit, the recess allowing the first principal surface to be larger than any of principal surfaces of the plurality of optical elements other than the first optical element, the recess being formed to a halfway position of a side surface of the first optical element, and
a reinforcing member is housed inside the recess.

9. The optical unit for endoscope according to claim 8, wherein the first principal surface is larger than the second principal surface.

10. The optical unit for endoscope according to claim 8, wherein the reinforcing member is made of a light-shielding material.

11. The optical unit for endoscope according to claim 9, wherein the reinforcing member is made of a resin material.

12. The optical unit for endoscope according to claim 9, wherein the reinforcing member is made of an inorganic material film or a plated film.

13. The optical unit for endoscope according to claim 8, wherein an image pickup device is further laminated, the image pickup device including, on a light-receiving surface, a light-receiving portion that receives light whose image is formed by the plurality of optical elements.

14. The optical unit for endoscope according to claim 13, wherein a size of the light-receiving surface of the image pickup device is smaller than a size of the second principal surface of the first optical element.

15. An endoscope including an optical unit for endoscope at a distal end portion of an insertion portion,
the optical unit for endoscope comprising:
a plurality of optical elements including a first optical element, the first optical element including a first principal surface as a light incident surface and a second principal surface opposed to the first principal surface, the first optical element having a base body made of a parallel flat glass, the plurality of optical elements being laminated and configured to form an image of light entered from the light incident surface,
wherein a recess is formed on a side surface of the optical unit, the recess allowing the first principal surface to be larger than any of principal surfaces of the plurality of optical elements other than the first optical element, the recess being formed to a halfway position of a side surface of the first optical element, and
a reinforcing member is housed inside the recess.
